(19) United States
(12) Patent Application Publication
(10) Pub. No.: US 2024/0299293 A1
(43) Pub. Date: Sep. 12, 2024

(54) Oral rinse, nasal spray and methods for prevention of COVID-19 by lowering viral load of COVID-19

(71) Applicant: Rucker Capital Advisors, G, NJ (US)

(72) Inventor: Keith D. Crawford, Westwood (OM)

(21) Appl. No.: 18/143,154

(22) PCT Filed: Nov. 9, 2021
(86) PCT No.: PCT/US2021/058578

§ 371 Date: Feb. 8, 2024

Related U.S. Application Data
(60) Provisional application No. 63/246,023, filed on Sep. 20, 2021.
(60) Provisional application No. 63/213,922, filed on Jun. 23, 2021.
(60) Provisional application No. 63/111,355, filed on Nov. 9, 2020.

(51) Int. Cl. A61K 9/00; A61K 31/05; A61K 31/603; A61K 31/731; A61K 31/755; A61K 45/06; A61P 31/14

(52) U.S. Cl. CPC A61K 9/006; A61K 31/05; A61K 31/603; A61K 31/731; A61K 31/755; A61K 45/06; A61P 31/14

(57) ABSTRACT

Compositions and methods are provided for a nasal spray or mouthwash formulation for prevention and amelioration of disease progression caused by a virus infection, the formulation including: an algae derivative; and a buffer. The method effectively bathes the mouth and throat tissues to decrease COVID-19 concentration and functionality; thereby preventing growth and spread of the virus and lower the viral load present in the lungs and in GI tract.

### RELATED APPLICATIONS

This application is a national stage filing of PCT application No.: PCT/US2021/58578 filed Nov. 9, 2021, entitled “Oral rinse, nasal spray and methods for prevention of COVID-19 by lowering viral load” which claims the benefit of priority of U.S. provisional application No. 63/111,355 filed Nov. 9, 2020; U.S. provisional application No. 63/213,922 filed Jun. 23, 2021, and U.S. provisional application No. 63/246,023 filed Sep. 20, 2021. The three provisional applications are entitled, “Oral rinse, nasal spray and methods for prevention of COVID-19 by lowering viral load of COVID-19” by inventor Dr. Keith D. Crawford. These applications are hereby incorporated by reference herein in their entireties.

### FIELD OF INVENTION

The present invention relates generally to oral rinse, mouthwash formulations and/or nasal spray formulations for prevention and amelioration of disease progression in a subject caused by a virus infection particularly, SARS-COV-2 virus causing Coronavirus disease 2019 (COVID-19). The formulation decreases viral inoculum and viral load of SARS-COV-2 virus in the subject and interferes with the binding, uptake, and/or fusion of the virus to an epithelial membrane of the upper respiratory tract mucosal tissue of the subject. Methods are provided herein for preventing a viral particle of the SARS-COV-2 virus from entering a host cell on a mucosal membrane of the subject.

### BACKGROUND

Viral entry into an orifice of host and into a cell of the host is the earliest stage of an infection in the viral life cycle. The virus encounters the host cell and introduces viral material into the host cell. Coronavirus (CoV) cell infection begins with viral entry, in which the viral particle recognizes a host cell receptor and fuses its membrane with the host cell membrane. Despite the variation among viruses, there are several shared generalities with respect to viral entry.

As of early November 2021, more than 248 million cases of SARS-COV-2 infection have been reported worldwide. A correlation between SARS-COV-2 viral load (VL) and the severity of illness and resulting mortality has been observed. A low initial SARS-COV-2 VL in the nasopharyngeal cavity is observed to be proportional to low probability of developing severe symptoms of COVID-19 and resulting mortality.

Early in the process of infection, the innate immune system detects a viral infection and mounts an “innate immune response”. The innate immune response slows the replication and spread of the virus, thereby keeping the infected subject alive until the acquired immune response is activated. For a new viral infection, the acquired immune response needs time from about two to about three weeks to stop the infection and generate immune memory.

In a COVID-19 infection, the immune system is able combat the infection in about 80% of the population who recover following a bout with a mild influenza-like illness. In older people, or people with immunodeficiencies, the activation of the acquired immune system is delayed. In cases with delayed activation of the acquired immune response, the innate immune response continues to increase as the virus replicates and spreads, and a “cytokine storm” occurs. The cytokine storm is difficult to manage clinically, requiring intensive care and treatment and highly increases risk of death.

The viral load that triggers a COVID-19 infection is unknown, however a massive dose of the virus leads to a massive innate immune response. Further, the innate immune response struggles to control the viral replication and does not allow adequate time for the acquired immune response to initiate. Uncontrolled cytokine storms lead to hemodynamic dysfunction and multi-organ failure. Decreasing the viral load allows the innate immune response to effectively control viral replication until the adaptive or acquired immune response is activated.

Therefore, there is a need to lower the viral load at the mucosal barrier. Further, there is a need for preventive measures, which decrease transmission and lower viral loads so that an individual has adequate time to mount an effective immune response.

### SUMMARY

An aspect of the invention described herein provides a nasal spray or mouthwash formulation for prevention and amelioration of disease progression caused by a virus infection, the formulation including: an iodine; an algae derivative; a buffer; and excipients. The virus is transmitted by respiratory droplets. The virus causing the infection includes: a coronavirus, an influenza virus, a parvovirus B19, a mumps virus, a rubella virus, a measles virus, and a respiratory syncytial virus.

In some embodiments the formulation essentially includes a mixture of at least one water soluble algae derivative. In some embodiments the formulation further includes a cosmetic buffer. In an embodiment of the formulation, the iodine is at least one selected from: povidone iodine, molecular iodine, elemental iodine, iodate derivative, iodide derivative, and periodate derivative. In an embodiment of the formulation, the algae derivative is selected from: a linear sulfated polysaccharide and a lectin. In an embodiment of the formulation, the alga derivative is sourced from at least one alga selected from: Euglenophyta, Chrysophyta, Pyrrophyta, Chlorophyta, Rhodophyta, Paeophyta, and Xanthophyta. In some embodiments, the algal derivative is selected from a carrageenan or a griffithsin. In an embodiment of the formulation, the carrageenan is selected from: kappa carrageenan, iota carrageenan, and lambda carrageenan.

An embodiment of the formulation may further include ethanol. An alternative embodiment of the formulation may not further include ethanol. An embodiment of the formulation further includes at least one essential oil selected from: eucalyptus oil, thyme oil, peppermint oil, clove oil, cinnamon oil, oregano oil, tea tree oil, pimento oil, rosemary oil, bergamot oil, lemongrass oil and lavender oil. An embodiment of the formulation further includes at least one essential oil compound selected from: eucalyptol, thymol, methyl salicylate, menthol, menthone, limonene, camphene, sabinene, and terpenes. An embodiment of the formulation further includes at least one sweetener selected from: sorbitol, xylitol, mannitol, erythritol, sodium saccharin, and lactitol. An embodiment of the formulation further includes at least one compound which is antibacterial or antiviral.

In an embodiment of the formulation, the compound is selected from: zinc chloride, benzoic acid, salicylic acid, lysozyme, lactoperoxidase, glucose oxidase, cetylpyridinium chloride, sodium fluoride, chlorhexidine gluconate, and hexetidine. An embodiment of the formulation further includes an antifungal compound selected from: sodium benzoate, potassium thiocyanate, and a mutanase.

An embodiment of the formulation further includes at least one flavoring agent selected from: peppermint, spearmint, cinnamon, cherry, apple, bubblegum, green tea, strawberry, blueberry, raspberry, lime, orange, and grape. An embodiment of the formulation further includes at least one coloring agent imparting a color selected from: blue, green, red, orange, and purple. An embodiment of the formulation further includes at least one surfactant selected from: Poloxamer 407, benzalkonium chloride, cetylpyridinium chloride, polyoxypropylene, and polyoxyethylene. An embodiment of the formulation further includes a humectant selected from: glycerin and sorbitol. In an embodiment of the formulation, the iodine is at a concentration of 0.001% to 1%. In an embodiment of the formulation, the algae derivative is at a concentration of 0.001% to 0.5%.

An aspect of the invention described herein provides a formulation for an oral and pharyngeal rinse, wash, or gargle rinse to prevent progression of infection of SARS-COV-2 virus causing Coronavirus disease 2019 (COVID-19), the formulation including: povidone iodine, iota carrageenan, thymol, eucalyptol, menthol, methyl salicylate, ethanol, and an orally acceptable buffer. An embodiment of the formulation may include: iota carrageenan, thymol, eucalyptol, menthol, methyl salicylate, ethanol, and an orally acceptable buffer and not include povidone iodine. An embodiment of the formulation further includes sorbitol, xylitol, zinc chloride and peppermint oil.

In an embodiment of the formulation, povidone iodine is at a concentration of 0.1% to 1%. In an embodiment of the formulation, iota carrageenan is at a concentration of 0.001% to 0.5%. In an embodiment of the formulation, thymol is at a concentration of 0.001% to 0.09%. In an embodiment of the formulation, menthol is at a concentration of 0.01% to 0.09%. In an embodiment of the formulation, eucalyptol is at a concentration of 0.05% to 0.1%. In an embodiment of the formulation, methyl salicylate is at a concentration of 0.01% to 0.1%. In an embodiment of the formulation, ethanol is at a concentration of 60% to 40%. In an embodiment of the formulation, the orally acceptable buffer is purified water.

An aspect of the invention described herein provides an oral or pharyngeal rinse, wash, or gargle formulation for prevention and treatment of SARS-COV-2 virus causing Coronavirus disease 2019 (COVID-19), the formulation includes: povidone iodine at a concentration of 0.001% to 1%, iota carrageenan at a concentration of 0.01% to 0.3%, thymol at a concentration of 0.01% to 0.09%, eucalyptol at a concentration of 0.05% to 0.1%, menthol at a concentration of 0.01% to 0.09%, methyl salicylate at a concentration of 0.01% to 0.1%, ethanol at a concentration of 20% to 40%, sorbitol at a concentration of 10% to 20%, xylitol at a concentration of 1% to 10%, zinc chloride at a concentration of 0.01% to 0.1%, and peppermint oil at a concentration of 0.01% to 0.1%. An embodiment of the formulation further includes an orally acceptable buffer.

An aspect of the invention described herein provides an oral or pharyngeal rinse, wash, or gargle formulation for preventing and treating infection of SARS-COV-2 virus, the formulation includes: povidone iodine at a concentration of 0.5%, iota carrageenan at a concentration 0.12%, thymol at a concentration of 0.064%, eucalyptol at a concentration of 0.0920%, menthol at a concentration of 0.0420%, methyl salicylate at a concentration of 0.0600%, ethanol at a concentration of 26.9%, sorbitol at a concentration of 14%, xylitol at a concentration of 5%, zinc chloride at a concentration of 0.08%, peppermint oil at a concentration of 0.04%, and purified water.

An aspect of the invention described herein provides a method for preventing a virus or a component of virus or a viral particle of a virus from entering a host cell on a mucosal membrane of a subject by decreasing a viral load of the virus, the method including: administering in a protocol of applying a polyinterferent composition, the composition including povidone iodine, iota carrageenan, thymol, eucalyptol, menthol, methyl salicylate, ethanol, and an acceptable buffer, to the mucosal membrane of the subject. Some embodiments of the method prevent the virus from entering the host cell by decreasing the ability of the virus to bind to receptors on the cell surface and decreasing the ability of the viral membrane to fuse to the host cell membrane thereby decreasing the viral inoculum

In an embodiment of the method, the protocol further includes at least one of the following steps selected from: irrigating nasal cavities of the subject with saline solution; rinsing oral and pharyngeal cavities of the subject with the polyinterferent composition; gargling mouth and throat of the subject with the polyinterferent composition; administering an antihistamine composition; and performing respiratory exercises. In an embodiment of the method, gargling further includes a regimen of at least two gargles a day for at least about 10 seconds, about 20 seconds, about 30 seconds, about 40 seconds, about 50 seconds, or about 60 seconds. In an embodiment of the method, gargling further includes contacting deep throat mucosal membranes with the polyinterferent composition.

An aspect of the invention described herein provides a polyinterferent formulation for prevention and amelioration of disease progression of SARS-COV-2 virus in an upper respiratory tract mucosal tissue of a subject, the formulation including: at least one compound selected from: povidone, carrageenan, griffithsin, thymol, eucalyptol, menthol, methyl salicylate, ethanol, sorbitol, xylitol, zinc chloride, peppermint oil, and purified water; the formulation does not include at least one compound selected from triclosan, chlorhexidene salts, cetylpyridinium chloride, and domiphen bromide; the formulation decreases viral inoculum and viral load of SARS-COV-2 virus in the subject and interferes with the binding, uptake, and/or fusion of the virus to an epithelial membrane of the upper respiratory tract mucosal tissue of the subject.

An aspect of the invention described herein provides a composition including an antimicrobial effective amount of uncomplexed molecular iodine (12), a source of iodate (103-) in an effective amount and a predetermined amount of an acid, such that the molar ratio of molecular iodine to iodate in the composition ranges from about 0.1 to about 25 to about 1.5 to about 5.0 and the concentration of acid in the composition is effective to provide a buffering pH ranging from about 1.5 to about 6.5, the composition provides a stable concentration of molecular iodine within the range of about 0.5 ppm to about 2500 ppm for a period of at least about 2 weeks to about 5 years.

An aspect of the invention described herein provides a method for reducing an amount of inoculum of a virus in a subject, the method including:
administering in a protocol of applying a polyinterferent composition, the composition including povidone iodine, iota carrageenan, thymol, eucalyptol, menthol, methyl salicylate, ethanol, and an acceptable buffer, to the mucosal membrane of the subject; the protocol including rinsing nasal cavities of the subject with the polyinterferent composition; gargling throat of the subject with the polyinterferent composition; administering an antihistamine composition; and performing respiratory exercises; and optionally reiterating the protocol thereby reducing the amount of inoculum of the virus in the subject.

An aspect of the invention described herein provides a nasal spray or a mouthwash or a chewing gum formulation for prevention and amelioration of disease progression caused by a SARS-COV-2 virus infection, the formulation includes: an algal derivative; and at least one of a buffer or an excipient.

In an embodiment of the formulation, the algal derivative is a carrageenan. In an embodiment of the formulation, the carrageenan is selected from: kappa carrageenan, iota carrageenan, and lambda carrageenan. An embodiment of the formulation further includes ethanol, for example denatured ethanol or 200 proof distilled ethanol.

An embodiment of the formulation further includes at least one essential oil selected from: eucalyptus oil, thyme oil, peppermint oil, clove oil, cinnamon oil, oregano oil, tea tree oil, pimento oil, rosemary oil, bergamot oil, lemongrass oil and lavender oil. An embodiment of the formulation further includes at least one essential oil compound selected from: eucalyptol, thymol, methyl salicylate, menthol, menthone, limonene, camphene, sabinene, and a terpene. An embodiment of the formulation further includes at least one sweetener selected from: sorbitol, xylitol, mannitol, erythritol, sodium saccharin, and lactitol. An embodiment of the formulation further includes water.

An aspect of the invention described herein provides a nasal spray or mouthwash or chewing gum formulation for prevention and amelioration of disease progression caused by a SARS-COV-2 virus infection, the formulation includes: thymol, menthol, eucalyptol, methyl salicylate, ethanol, sorbitol, xylitol, iota-carrageenan, peppermint oil, and water.

An aspect of the invention described herein provides an oral rinse formulation for preventing and treating infection of SARS-COV-2 virus, the formulation including: iota carrageenan at a concentration 0.06%, thymol at a concentration of 0.06%, eucalyptol at a concentration of 0.0920%, menthol at a concentration of 0.0420%, methyl salicylate at a concentration of 0.0600%, ethanol at a concentration of 26.9%, sorbitol at a concentration of 14%, xylitol at a concentration of 5%, peppermint oil at a concentration of 0.04%, and purified water.

An aspect of the invention described herein provides a method for preventing a viral particle of a SARS-COV-2 virus from entering a host cell on a mucosal membrane of a subject by decreasing viral load, the method including: administering in a protocol of applying a polyinterferent composition, the composition including iota carrageenan, thymol, eucalyptol, menthol, methyl salicylate, ethanol, and an acceptable buffer, to the mucosal membrane of the subject.

In an embodiment of the method, the protocol further includes at least one of step selected from: irrigating nasal cavities of the subject with saline solution; rinsing oral and pharyngeal cavities of the subject with the polyinterferent composition; gargling mouth and throat of the subject by contacting deep throat mucosal membranes with the polyinterferent composition; administering an antihistamine composition; and performing respiratory exercises. In an embodiment of the method, the polyinterferent composition is in at least one form selected from: a mouthwash, a nasal spray, and a chewing gum.

An aspect of the invention described herein provides a method for reducing a SARS-CoV-2 viral inoculum in a subject exposed to the virus, the method including: administering in a protocol of applying a polyinterferent composition, the composition including iota carrageenan, thymol, eucalyptol, menthol, methyl salicylate, ethanol, and an acceptable buffer, to the mucosal membrane of the subject; the protocol including irrigating nasal cavities of the subject with saline solution; rinsing oral and pharyngeal cavities of the subject with the polyinterferent composition; gargling mouth and throat of the subject by contacting deep throat mucosal membranes with the polyinterferent composition; and optionally further including administering an antihistamine composition; and/or performing respiratory exercises; and optionally reiterating the protocol thereby reducing the amount of inoculum of the virus in the subject.

An aspect of the invention described herein provides a chewing gum formulation for prevention and amelioration of disease progression caused by a SARS-COV-2 virus infection, the formulation includes: thymol, menthol, eucalyptol, methyl salicylate, ethanol, sorbitol, xylitol, iota-carrageenan, peppermint oil, and water.

In an embodiment of the formulation, the chewing gum includes an exterior surface and an interior liquid. In an embodiment of the formulations, the exterior surface is hard or soft.

An aspect of the invention described herein provides a method for preventing a viral particle of a SARS-COV-2 virus from entering a host cell on a mucosal membrane of a subject by decreasing viral inoculum, the method including: administering a chewing gum including an exterior surface encasing a liquid polyinterferent composition, the composition including iota carrageenan, thymol, eucalyptol, menthol, methyl salicylate, ethanol, and an acceptable buffer; breaking the exterior surface of the chewing gum by the subject biting the chewing gum to release the liquid polyinterferent composition into an oral cavity of the subject; gargling mouth and throat of the subject by contacting deep throat mucosal membranes with the polyinterferent composition; and expectorating the polyinterferent composition thereby decreasing viral load in the subject.

An aspect of the invention described herein provides a nasal spray formulation for prevention and amelioration of disease progression caused by a SARS-COV-2 virus infection, the formulation includes: iota-carrageenan, sodium chloride, xylitol, benzyl alcohol, potassium sorbate, citric acid, and water.

An aspect of the invention described herein provides a nasal rinse formulation for preventing and treating infection of SARS-COV-2 virus, the formulation includes: iota carrageenan at a concentration 0.06%, sodium chloride at a concentration of 1.5%, benzyl alcohol at a concentration of 0.2%, xylitol at a concentration of 5%, potassium sorbate at a concentration of 0.12%, citric acid at a concentration of 0.025%, and purified water.

An aspect of the invention described herein provides a method for preventing a viral particle of a SARS-COV-2 virus from entering a host cell on a mucosal membrane of a subject by decreasing viral load, the method includes: administering in a protocol of applying a polyinterferent composition, the composition including iota-carrageenan, sodium chloride, xylitol, benzyl alcohol, potassium sorbate, citric acid, and an acceptable buffer, to the mucosal membrane of the subject.

An aspect of the invention described herein provides a nasal rinse formulation for decreasing viral load of a SARS-COV-2 virus in a subject, the formulation including: iota carrageenan at a concentration 0.06%, sodium chloride at a concentration of 1.5%, benzyl alcohol at a concentration of 0.2%, xylitol at a concentration of 5%, potassium sorbate at a concentration of 0.12%, citric acid at a concentration of 0.025%, and purified water.

An aspect of the invention described herein provides a nasal rinse formulation for prevention and amelioration of disease progression caused by a virus infection, the formulation includes: an algal derivative, a salt, an alcohol, and a buffer. In an embodiment of the nasal rinse formulation, the algal derivative is iota carrageenan at a concentration from about 0.01% to about 0.1%. In an embodiment of the nasal rinse formulation, the salt is sodium chloride at a concentration from about 0.5% to about 2%. In an embodiment of the nasal rinse formulation, the alcohol is benzyl alcohol at a concentration from about 0.01% to about 0.9%.

An aspect of the invention described herein provides a kit for preventing a viral particle from entering a host cell on a mucosal membrane of a subject by decreasing a viral load, the kit including: a mouthwash comprising an algal derivative, an alcohol, and at least one excipient; a nasal rinse comprising an algal derivative, a salt, and at least one excipient; and instructions for use. In an embodiment of the kit, the instructions further include a protocol of applying the mouthwash and the nasal rinse to the mucosal membrane of the subject at least once a day. The mucosal membranes of the subject include the mucosal membranes of the nasal cavity, the oral cavity, and the oropharyngeal cavity. In some embodiments, the protocol includes: irrigating nasal cavities of the subject with the nasal rinse; rinsing oral and pharyngeal cavities of the subject with the mouthwash; and gargling mouth and throat of the subject by contacting deep throat mucosal membranes with the mouthwash. In some embodiments, the nasal cavities are irrigated using a nasal irrigator, for example a nasal rinse bottle, a neti pot, or a nasal irrigation system. In an embodiment the nasal irrigator is an electronic nasal irrigator having a powered suction to remove the nasal rinse from the nasal cavities. In some embodiments of the kit, the protocol further includes at least one of: administering an antihistamine composition, and performing respiratory exercises. In some embodiments of the kit, the viral particle is from a SARS-COV-2 virus.

An embodiment of the kit further includes a test for COVID-19, the test optionally having electronic connectivity to a handheld device, for example the test is Bluetooth enabled. In some embodiments of the kit, the test is administered to the subject and a COVID-19 test result is obtained. In some embodiments, the COVID-19 test result is transmitted to the handheld device and/or to a health care provider. Some embodiments of the kit further include at least one of: a chlorhexidine gluconate composition, and a dexamethasone composition. In some embodiments the chlorhexidine gluconate composition and/or the dexamethasone composition is a mouthwash solution. The chlorhexidine mouthwash solution and/or the dexamethasone mouthwash solution is applied to the mucosal membrane of the subject at least

### BRIEF DESCRIPTION OF DRAWINGS

FIG. 1 is a schematic drawing of anatomy of the nasopharynx, the oropharynx and the laryngopharynx.

FIG. 2 is a graph of expression levels of ACE2 on tongue, floor of mouth, base of tongue and other sites.

### DETAILED DESCRIPTION

To assist in understanding the following description of various embodiments, specific definitions are provided below. Unless the surrounding text clearly indicates the opposite intention, the following definitions are intended here:

“Anti-microbial agent” means a substance that has activity to reduce the number and viability for function of one or more strains of bacteria, viruses, fungi, virus-like bodies, prions, and the like.

“Active antiviral agent” means an agent that inhibits any step of viral replication, for example, an agent that inhibits the initial step in the viral life cycle of binding of a virus to a host cell, or for example, an agent capable of binding to betacorona virus spike protein receptors.

“Biologically compatible” means that a significant long-term adverse effect on the surface of or in the body of a mammalian species is not observed.

“Infectious dose” means the number of viral particles a subject is exposed to at the start of an infection.

“In-hospital infection” means a localized or systemic infection that has no evidence of incubation and is clinically evident, most often within 48 hours of hospitalization.

“Microbials” refers to all types of microorganisms, including but not limited to bacteria, viruses, fungi, virus-like bodies, prions, and the like.

“Minimal infective dose” is defined as the lowest number of viral particles or virions that cause an infection in 50% of individuals (or ‘the average person’).

“Viral load” is determined after a subject is infected with a virus, and the subject's cells replicates the virus. The total amount of amount of virus or virions or viral nucleic acid replicated by a subject and being carried by the subject is referred to as their ‘viral load’.

“Variolation” is the level of inoculum below which the immune system is overwhelmed resulting in serious illness.

“Oral polyinterferent composition” is a product which, in its normal use, is not intentionally swallowed for the purpose of systemic administration of therapeutic agents, but is retained in the oral and oropharyngeal cavities for a sufficient time to impact viruses.

“Oral cavity” refers to the mouth and includes the lips, the lining inside the cheeks and lips, the front two thirds of the tongue, the upper and lower gums, the floor of the mouth under the tongue, the bony roof of the mouth, and the small portion behind the wisdom teeth.

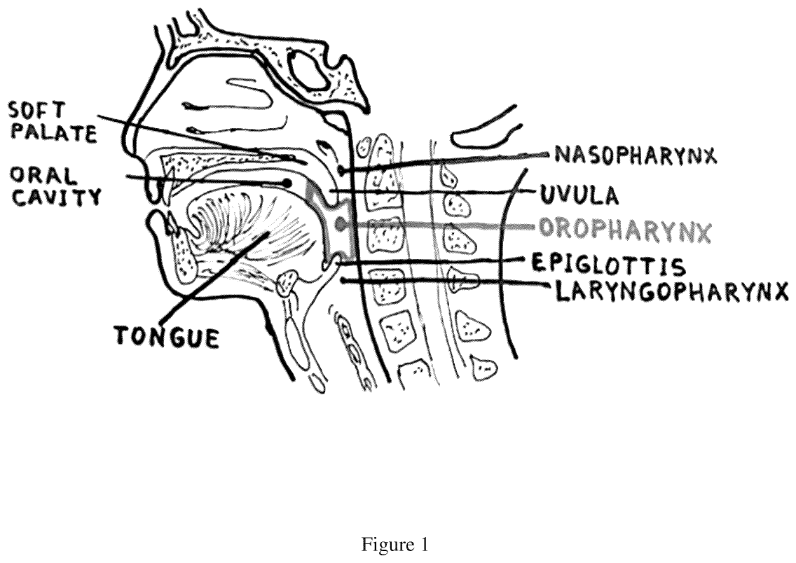

“Oropharynx” is the part of the throat just behind the mouth. It starts where the oral cavity stops. It includes the base of the tongue (the back third of the tongue), the soft palate (the back part of the roof of the mouth), the tonsils, and the side and back walls of the throat. See FIG. 1.

“Mouthwash” or “mouth rinse” or “oral rinse” or “mouth bath” is a liquid which is held in the mouth passively or swilled around the mouth by contraction of the perioral muscles and/or movement of the head, and may be gargled, in which the head is tilted back and the liquid is bubbled at the back of the mouth. Generally, mouthwashes are antiseptic solutions intended to reduce the microbial load in the oral cavity, although mouthwashes are prescribed for other reasons such as for their analgesic, anti-inflammatory or anti-fungal action. Additionally, some rinses act as saliva substitutes to neutralize acid and keep the mouth moist in xerostomia (dry mouth). Cosmetic mouth rinses temporarily control or reduce bad breath and leave the mouth with a pleasant taste.

“Molecular iodine” or “uncomplexed molecular iodine” refers to diatomic iodine, which is a molecule comprised of 2 iodine atoms and is represented by the chemical symbol I₂ (CAS Registry Number: 7553-56-2).

“Inoculum” refers to the amount of microbial introduced into the body of a subject.

“Viral entry” is the earliest stage of infection in the viral life cycle, as the virus comes into contact with the host cell and introduces viral material into the cell. The process for viral entry is dependent on the type of virus. A virus with a naked capsid enters the cell by attaching to the attachment factor located on a host cell, making a hole in the membrane of the host cell and inserting the viral genome. An enveloped virus attaches to an attachment factor located on the surface of the host cell, the virus membrane and the host cell membrane fuse together allowing the virus particles to enter the susceptible host cell. A susceptible host cell is a cell which expresses a compatible binding receptor. The receptors on the viral envelope connect to complementary receptors on the cell membrane of the host cell. The attachment causes the two membranes to remain in mutual proximity, favoring further interactions between surface proteins. The attachment of the two membranes is the first requisite that must be satisfied before a cell becomes infected. Viruses that exhibit this behavior include numerous enveloped viruses such as HIV, Herpes Simplex, and SARS-CoV-19.

The viral particles enter the host cell that is covered by a phospholipid bilayer which is a natural barrier of the host cell. The process by which this barrier is breached depends upon the type of virus. There are three types of viral particle entry: Membrane fusion, in which the cell membrane is punctured and connected with the unfolding viral envelope; Endocytosis in which the host cell absorbs the viral particle by the process of endocytosis, essentially engulfing the virus similar to a food particle; and Viral penetration in which the viral capsid or genome is injected into the host cell's cytoplasm.

The most well-known type of viral entry is by membrane fusion. In viruses with a viral envelope, viral receptors attach to the receptors on the surface of the cell and secondary receptors initiate the puncture of the membrane or fusion with the host cell. Following attachment, the viral envelope fuses with the host cell membrane, resulting in the entry of viral particles.

SARS-COV-2 virus cell infection begins with viral entry, in which the viral particle recognizes a host cell receptor and fuses its membrane with the host cell membrane. Belouzard, S. et al., 2009 Proc. Natl. Acad. Sci. 106, 5871-5876. The steps of recognition of the host cell receptor and fusion of the membrane are mediated by the coronavirus spike (S) protein. In addition to mediating entry, the S protein is the principal antigenic determinant and the target of neutralizing antibodies. Walls, A. C. et al., 2020. Cell 180, 281-292. Therefore, S protein is a valuable target in vaccine and antiviral efforts. Du et al., Expert Opin. Ther. Targets 21, 131-143.

As of early November 2021, more than 248 million cases of SARS-COV-2 infection have been reported worldwide. Viral load measurements from tissue samples are indicative of active virus replication and are routinely used to monitor severe viral respiratory tract infections including clinical progression, response to treatment, cure, and relapse. Rainer, T H. et al., 2007 Eur J Clin Microbiol Infect Dis 26:121-9; Zaki A M. et al., 2012 N Engl J Med; 367:1814-20; Memish Z A. et al., 2014 J Infect Dis 210:1590-4.

A correlation between SARS-COV-2 viral load (VL) and the severity of illness and resulting mortality has been observed. A low initial SARS-COV-2 VL in the nasopharyngeal cavity is proportional to low probability of developing severe symptoms of COVID-19 and resulting mortality.

The viral loads in samples from the upper respiratory tract of 18 patients with coronavirus disease 2019 (covid-19, an infectious disease caused by SARS-COV-2) were observed to be equal in asymptomatic patients and symptomatic patients. Zou L. et al., 2020 N Engl J Med; 382:1177-9. However, the viral load dynamics in lower respiratory tract and other tissue samples and the relation between viral load and disease severity is important for the formulation of disease control strategies and clinical treatment.

### Influenza Viruses

There are four types of influenza viruses: A, B, C and D. Human influenza A and B viruses cause seasonal epidemics of disease (known as the flu season) almost every winter in the United States. Influenza A viruses are the only influenza viruses known to cause flu pandemics, i.e., global epidemics of flu disease. A pandemic occurs if a new and very different influenza A virus emerges that both infects people and has the ability to spread efficiently between people. Influenza type C infections generally cause mild illness and are not thought to cause human flu epidemics. Influenza D viruses primarily affect cattle and are not known to infect or cause illness in people.

Influenza A viruses are divided into subtypes based on two proteins on the surface of the virus: hemagglutinin (H) and neuraminidase (N). There are 18 different hemagglutinin subtypes and 11 different neuraminidase subtypes (H1 through H18 and N1 through N11, respectively). 198 different influenza A subtype combinations are potentially possible, however, only 131 subtypes have been detected in nature. Current subtypes of influenza A viruses that routinely circulate in people include: A(H1N1) and A(H3N2). Influenza A subtypes are further sub-divided into different genetic “clades” and “sub-clades.”

Clades and sub-clades are alternatively called “groups” and “sub-groups,” respectively. An influenza clade or group is a further subdivision of influenza viruses (beyond subtypes or lineages) based on the similarity of their HA gene sequences. Dividing viruses into clades and subclades allows flu experts to track the proportion of viruses from different clades in circulation. Note that clades and sub-clades that are genetically different from others are not necessarily antigenically different (i.e., viruses from a specific clade or sub-clade may not have changes that impact host immunity in comparison to other clades or sub-clades).

Currently circulating influenza A(H1N1) viruses are related to the pandemic 2009 H1N1 virus that emerged in the spring of 2009 and caused a flu pandemic. This virus, scientifically called the “A(H1N1)pdm09 virus,” and more generally called “2009 H1N1,” has continued to circulate seasonally. These H1N1 viruses have undergone relatively small genetic changes and changes to their antigenic properties over time.

Of all the influenza viruses that routinely circulate and cause illness in people, influenza A(H3N2) viruses tend to change more rapidly, both genetically and antigenically. Influenza A(H3N2) viruses have formed many separate, genetically different clades in recent years that continue to co-circulate.

Influenza B viruses are not divided into subtypes, but instead are further classified into two lineages: B/Yamagata and B/Victoria. Influenza B viruses are further classified into specific clades and sub-clades. Influenza B viruses generally change more slowly in terms of their genetic and antigenic properties than influenza A viruses, especially influenza A(H3N2) viruses. Influenza surveillance data from recent years shows co-circulation of influenza B viruses from both lineages in the United States and around the world. However, the proportion of influenza B viruses from each lineage that circulate varies by geographic location.

CDC follows an internationally accepted naming convention for influenza viruses. This convention was accepted by WHO in 1979 and published in February 1980 in the Bulletin of the World Health Organization, 58(4):585-591 (1980) (see A revision of the system of nomenclature for influenza viruses: a WHO Memorandum). The approach uses the following components:
The antigenic type (e.g., A, B, C, D)
The host of origin (e.g., swine, equine, chicken, etc.). For human-origin viruses, no host of origin designation is given. Note the following examples:
(Duck example): avian influenza A(H1N1), A/duck/Alberta/35/76
(Human example): seasonal influenza A(H3N2), A/Perth/16/2019
Geographical origin (e.g., Denver, Taiwan, etc.)
Strain number (e.g., 7, 15, etc.)
Year of collection (e.g., 57, 2009, etc.)

For influenza A viruses, the hemagglutinin and neuraminidase antigen descriptions are provided in parentheses (e.g., influenza A(H1N1) virus, influenza A(H5N1) virus)

The 2009 pandemic virus was assigned a distinct name: A(H1N1)pdm09 to distinguish it from the seasonal influenza A(H1N1) viruses that circulated prior to the pandemic.

Strains of influenza viruses that normally circulate in swine (pigs) that infect humans are called variant viruses and are designated with a letter ‘v’ (e.g., an A(H3N2)v virus).

### Respiratory Syncytial Virus (RSV)

Respiratory syncytial virus, or RSV, is a common respiratory virus that causes mild, cold-like symptoms. Most people recover in a week or two, but RSV may develop into serious illnesses, especially for infants and older adults. RSV is the most common cause of bronchiolitis (inflammation of the small airways in the lung) and pneumonia (infection of the lungs) in children younger than 1 year of age in the United States. RSV is a droplet infection which is spread by an infected person upon coughing and sneezing; getting virus droplets from a cough or sneeze in your eyes, nose, or mouth; and touching a surface that has the virus on it, like a doorknob, and then touching the face before washing your hands.

People infected with RSV are usually contagious for 3 to 8 days. However, some infants, and people with weakened immune systems, continue to spread the virus even after the symptoms have abated for as long as 4 weeks. Children are often exposed to and infected with RSV outside the home, such as in school or child-care centers. Children then transmit the virus to other members of the family. RSV survives for many hours on hard surfaces such as tables and crib rails. It typically lives on soft surfaces such as tissues and hands for shorter amounts of time.

People of any age may get a subsequent RSV infection, but infections later in life are generally less severe. People at highest risk for severe disease include: premature infants, young children with congenital (from birth) heart or chronic lung disease, young children with compromised (weakened) immune systems due to a medical condition or medical treatment, adults with compromised immune systems, and older adults with underlying heart or lung disease.

In the United States and other areas with similar climates, RSV infections generally occur during fall, winter, and spring. The timing and severity of RSV circulation in a given community can vary from year to year.

### Measles

Measles is a highly contagious infectious disease caused by measles virus. Symptoms usually develop from about 10 to about 12 days after exposure to an infected person and last from about 7 to about 10 days. Initial symptoms typically include fever, often greater than 104° F., cough, runny nose, and inflamed eyes. Small white spots known as Koplik's spots may form inside the mouth two or three days after the start of symptoms. A red, flat rash which usually starts on the face and then spreads to the rest of the body typically begins three to five days after the start of symptoms.

Common complications include diarrhea, middle ear infection, and pneumonia. These complications occur due to measles-induced immunosuppression. Less common complications include seizures, blindness, or inflammation of the brain.

Measles is an airborne disease which spreads easily from one person to the next through the coughs and sneezes of infected people. It also spreads through direct contact with mouth or nasal secretions. Measles is extremely contagious, and patients are infectious to others from about four days before the rash appears to about four days after the start of the rash appears.

### Coronaviruses

Coronaviruses are classified as a family within the Nidovirales order, viruses that replicate using a nested set of mRNAs (“nido-” or “nest”). The coronavirus subfamily is further classified into four categories: alpha, beta, gamma, and delta coronaviruses. The human coronaviruses (HCoVs) are classified within two of these genera: alpha coronaviruses (HCoV-229E and HCoV-NL63) and beta coronaviruses (HCoV-HKU1, HCoV-OC43, Middle East respiratory syndrome coronavirus [MERS-COV], the severe acute respiratory syndrome coronavirus [SARS-COV]), and SARS-COV-2. Chan J F. et al., 2015 Clin Microbiol Rev; 28:465; International Committee on Taxonomy of Viruses.

### Viral Composition of Coronaviruses

Coronaviruses are medium-sized enveloped positive-stranded RNA viruses. The coronaviruses derive their name from their characteristic crown-like appearance in electron micrographs. McIntosh K. et al., 1967 Proc Natl Acad Sci USA; 57:933; Masters P S. et al., Lippincott Williams & Wilkins, a Wolters Kluwer business, Philadelphia 2013. Vol 2, p.825.

Coronaviruses have the largest known viral RNA genomes, with a length of 27 to 32 kb. The membrane is studded with glycoprotein spikes and surrounds the genome, which is encased in a nucleocapsid that is helical in its relaxed form and assumes a roughly spherical shape in the virus particle. Replication of viral RNA occurs in the host cytoplasm by a unique mechanism in which RNA polymerase binds to a leader sequence and then detaches and reattaches at multiple locations, allowing for the production of a nested set of mRNA molecules with common 3′ ends. The genome encodes four or five structural proteins, particularly, proteins known as S, M, N, HE, and E. Strains known as HCoV-229E, HCoV-NL63, and the SARS coronavirus possess four genes that encode the S, M, N, and E proteins, respectively, whereas the strains HCoV-OC43 and HCoV-HKU1 also contain a fifth gene that encodes the HE protein. McIntosh K, Peiris J S M. Coronaviruses. In: Clinical Virology, 3rd ed, Richman D D, Whitley R J, Hayden F G (Eds), ASM Press, Washington, D C 2009. p.1155.

The spike (S) protein projects through the viral envelope and forms the characteristic spikes in the coronavirus “crown.” It is heavily glycosylated, probably forms a homotrimer, and mediates receptor binding and fusion with the host cell membrane. The major antigens that stimulate neutralizing antibody, as well as important targets of cytotoxic lymphocytes, are on the S protein. McIntosh K, et al. Proc Natl Acad Sci USA 1967; 57:933.

The membrane (M) protein has a short N-terminal domain that projects on the external surface of the envelope and spans the envelope three times, leaving a long C terminus inside the envelope. The M protein plays an important role in viral assembly. Masters P S, Perlman S. Coronaviridae. In: Fields Virology, 6th ed, Knipe D M, Howley P M, Cohen J I, et al (Eds), Lippincott Williams & Wilkins, a Wolters Kluwer business, Philadelphia 2013. Vol 2, p.825.

The nucleocapsid protein (N) associates with the RNA genome to form the nucleocapsid. The nucleocapsid is involved in the regulation of viral RNA synthesis and interacts with M protein during virus budding. Enjuanes L, et al. Development of protection against coronavirus induced diseases. A review. Adv Exp Med Biol 1995; 380:197; Masters P S. et al., Adv Exp Med Biol 2006; 581:163. Cytotoxic T lymphocytes recognizing portions of the N protein have been identified. Kuo L, Masters P S. J Virol 2002; 76:4987.

The hemagglutinin-esterase glycoprotein (HE) is found only in the betacoronaviruses, HCoV-OC43 and HKU1. The hemagglutinin moiety binds to neuraminic acid on the host cell surface, permitting initial adsorption of the virus to the membrane. The esterase cleaves acetyl groups from neuraminic acid. The HE genes of coronaviruses have sequence homology with influenza C HE glycoprotein and reflect an early recombination between the two viruses. Perlman S. Adv Exp Med Biol 1998; 440:503.

The small envelope (E) protein leaves its C terminus inside the envelope and then either spans the envelope or bends around and projects its N terminus internally. The function of the E protein is not known, although, in the SARS-COV, the E protein with M and N are required for proper assembly and release of the virus. Luytjes W. et al., Virology 1988; 166:415; Siu Y L. et al., J Virol 2008; 82:11318.

### Viral Serotypes

Coronaviruses are widespread among birds and mammals, with bats being host to the largest variety of genotypes. Anthony S J, et al., Virus Evol 2017; 3. Animal and human coronaviruses are classified into four distinct genera. Six coronavirus serotypes have been associated with disease in humans: HCOV-229E, HCoV-NL63, HCoV-OC43, HCoV-HKU1, SARS-COV, SARS-COV-2, and MERS-COV.

The alphacoronavirus genus includes two human virus species, HCoV-229E and HCoV-NL63. HCoV-229E utilizes aminopeptidase N (APN) as its major receptor which is similar to several animal alphacoronaviruses. Yeager C L et al., Nature 1992; 357:420. In contrast, HCoV-NL63, similar to SARS-COV and SARS-COV-2 (betacoronaviruses), uses angiotensin-converting enzyme-2 (ACE-2). Hofmann H, et al. Proc Natl Acad Sci USA 2005; 102:7988.

Important animal alphacoronaviruses are transmissible gastroenteritis viruses of pigs and feline infectious peritonitis viruses. Bat coronaviruses are among the alphacoronaviruses. Two of the non-SARS human species of the betacoronavirus genus, HCoV-OC43 and HCoV-HKU1, have hemagglutinin-esterase activity and utilize sialic acid residues as receptors. Vlasak R et al., Proc Natl Acad Sci USA 1988; 85:4526. The betacoronavirus genus also contains several bat viruses, such as MERS-COV, SARS-COV, and SARS-COV-2, although these viruses are genetically distant from HCoV-OC43 and HCoV-HKU1. Zaki A M et al., N Engl J Med 2012; 367:1814; Centers for Disease Control and Prevention (CDC). Severe respiratory illness associated with a novel coronavirus—Saudi Arabia and Qatar, 2012. MMWR Morb Mortal Wkly Rep 2012; 61:820. Important animal betacoronavirus include strains such as mouse hepatitis virus, a laboratory model for both viral hepatitis and demyelinating central nervous system disease, and bovine coronavirus, a diarrhea-causing virus of cattle. Bovine coronavirus is highly similar to HCoV-OC43 that the two viruses have been merged into a single species termed betacoronavirus 1. Carstens E B. Arch Virol 2010; 155:133. HCoV-OC43 is believed to have jumped from one animal host to the other as recently as 1890. Vijgen L, et al. J Virol 2005; 79:1595.

The gammacoronavirus genus contains primarily avian coronaviruses, the most prominent of which is avian infectious bronchitis virus (AIBV), an important veterinary pathogen causing respiratory and reproductive tract disease in chickens. The deltacoronavirus genus contains recently discovered avian coronaviruses found in several species of songbirds.

None of the common cold human coronaviruses (HCoV-OC43, HCoV-NE63, HCoV-HKU1, and HCoV-229E) have been found to conveniently replicate in cell or tissue culture, and, until recently, progress in their study was impeded by lack of progress with in vitro culture of these strains. Both HCoV-229E and HCoV-OC43 were discovered in the 1960s and were shown in volunteer experiments to produce common colds in adults. McIntosh K et al. Proc Natl Acad Sci USA 1967; 57:933; Hamre D, Procknow J J. Proc Soc Exp Biol Med 1966; 121:190; Bradburne A F. Arch Gesamte Virusforsch 1970; 31:352; Bradburne A F et al., Br Med J 1967; 3:767. Studies in the 1970s and 1980s linked the common cold coronaviruses to about one-third of upper respiratory tract infections during winter outbreaks, 5 to 10 percent of overall colds in adults, and some proportion of lower respiratory illness in children.

Until the emergence of SARS in 2002, the lack of development of molecular diagnostic methods delayed acquisition of further information about this class of viruses. After SARS in 2002, HCoV-NL63 and HCoV-HKU1 were quickly discovered and observed to have worldwide distribution. Esper F, et al., J Infect Dis 2005; 191:492; Fouchier R A, et al., Proc Natl Acad Sci USA 2004; 101:6212; van der Hoek L, et al., Nat Med 2004; 10:368; Woo P C. et al., J Virol 2005; 79:884. The polymerase chain reaction is used for the diagnosis of each of the four human coronaviruses, and this technique has allowed substantial investigation into their epidemiology and pathogenicity.

### Treatment and Prevention

There is currently no treatment recommended for common cold coronavirus infections except for supportive care as needed. Chloroquine, which has potential antiviral activity against SARS-COV, has been shown to have similar activity against HCoV-229E in cultured cells and against HCoV-OC43 both in cultured cells and in a mouse model. However, there have been no studies of efficacy in humans. Kono M, et al., Antiviral Res 2008; 77:150; Keyaerts E et al., Antimicrob Agents Chemother 2009; 53:3416.

Preventive measures for coronaviruses are similar to preventive measure for rhinovirus infections, which consist of handwashing and careful disposal of materials infected with nasal secretions. The use of surface disinfectants is also an important tool in infection control, since coronaviruses survive for one or more days after drying on surfaces such as stainless steel, plastic, or cloth. Otter J A, et al., J Hosp Infect 2016; 92:235.

The efficacy of various disinfectants on human coronaviruses, including CoV-229E and SARS-COV, and animal coronaviruses (e.g., mouse hepatitis virus and transmissible gastroenteritis virus of pigs) was examined. These viruses (both in suspension and dried on surfaces) were characterized as being highly susceptible to 70% ethanol, with a reduction of viability by greater than 3 log (more than thousand-fold in titer of viable viruses) within seconds. Likewise, hexachlorophene, 2% glutaraldehyde and 1% povidone-iodine each produced satisfactory elimination rates. The susceptibility of coronaviruses to 6% sodium hypochlorite (the active agent in bleach) solutions is observed to be variable, however satisfactory level of elimination of virus was achieved with concentrations of a dilution of about 1:40 or at a higher concentration. Coronaviruses were observed to be eliminated by benzalkonium chloride or chlorhexidine only if 70% ethanol is present in the solution. Geller C, et al., Viruses 2012; 4:3044; Sattar S A, et al., Epidemiol Infect 1989; 102:49; Hulkower R L, et al., Am J Infect Control 2011; 39:401; Dellanno C, et al., Am J Infect Control 2009; 37:649; Cao J, et al., Antiviral Res 2015; 114:1.

There has been little interest in developing vaccines for the common cold coronaviruses for several reasons. Four separate species have been described and there is evidence within at least one of these species of clinically significant antigenic variation. Reed S E. J Med Virol 1984; 13:179. In addition, vaccine enhancement of disease has been shown for one animal coronavirus, particularly, feline coronavirus. Hypersensitivity was observed to be induced in some animals by prior exposure to a vaccine containing the S protein, with the production of an immunologically mediated severe disease (feline infectious peritonitis) upon reinfection with a coronavirus. Vennema H, et al., J Virol 1990; 64:1407.

### Viral Load

Viruses are self-replicating, which means an infection starts with a small number of particles (the dose). The actual minimum number varies between different viruses and the individual. The minimum infectious dose of COVID-19 is unknown, but it is presumed around a hundred virus particles.

The dose reaches the respiratory tract and infects one or two cells which are re-programmed to multiply and produce new viruses within 12-24 hours. For COVID-19, the dose and the time required to multiply is unknown. The new viruses infect nearby cells which includes cells of the immune defense system, possibly compromising the cells, and the entire process repeats.

Early in infection, the innate immune system detects a viral infection and mounts an “innate immune response”. This is not a virus-specific, “acquired immune response” which is medicated by antibodies. Rather innate immune response is a broad, non-specific, anti-viral response characterized by interferon and cytokines which are small proteins that have the side effect of causing various symptoms such as fever, headaches, muscle pain, etc.

The innate immune response serves two purposes: to slow down the replication and spread of the virus, thereby keeping the infected subject alive until the acquired immune response is activated. For a new viral infection, the acquired immune response takes from 2 to 3 weeks to stop and finally clear the infection and generating immune memory. Immune memory allows a faster response if an individual is infected in the future. The process serves the basis of the expected immunity in survivors and in vaccinated subjects.

In COVID-19, the two arms of the immune system which are the innate and acquired immune systems, function well in about 80% of the population, and these individuals recover from a more or less mild influenza-like illness. In older people, and in people with immunodeficiencies, the activation of the acquired immune system may be delayed.

Therefore, the virus continues to replicate and spread in the body. Another role of the acquired immune system is to stand-down, or to down-regulate or repress the innate immune system. Therefore, in cases with delayed activation of the acquired immune response, the innate immune response continues to increase as the virus replicates and spreads. A mechanism of the innate immune response is to cause “inflammation”. The inflammation is useful in containing the virus early in an infection but results in widespread damage of uninfected tissue which is known as “bystander effect”. If the damage of uninfected tissue continues and is uncontrolled, a condition named “cytokine storm” occurs. The cytokine storm is difficult to manage clinically, requiring intensive care and treatment and highly increases risk of death.

These scenarios describe immune responses following infection with a typical exposure to virus, in patients who make a recovery, in patients who require intensive care and in patients (mainly elderly and/or immunosuppressed) who might succumb. Patients with other comorbidities most likely succumb due to additional stress of a compromised essential systems by virus and/or cytokine storm.

The viral load that triggers a COVID-19 infection is unknown, however if a subject receives a massive exposure to the virus (for example from bodily fluids of infected patients which may contain a million and may even contain as many as a hundred million viruses per ml), particularly through inhalation, such a viral load jump starts a viral infection, which leads to a massive innate immune response. Further, the innate immune response struggles to control the viral replication and does not allow adequate time for the acquired immune response to initiate. Uncontrolled cytokine storms lead to hemodynamic dysfunction and multi-organ failure.

To assist the innate immune response to contain the COVID-19 infection, there is a need to lower the viral load at the mucosal barrier at the point of initial entry. Decreasing the viral load allows the innate immune response to effectively control viral replication until the adaptive or acquired immune response is activated.

For many bacterial and viral pathogens, a minimal infective dose is known however, because SARS-COV-2 is a new pathogen the general infective dose is unknown. For SARS, the infective dose in mouse models was only a few hundred viral particles. Therefore, it is likely that an individual is infected and develops symptoms of COVID-19 by merely inhaling a few hundred of SARS-COV-2 particles. This relatively low infective dose could explain the extensive spread of the virus.

On the basis of previous work on SARS and MERS coronaviruses, it is known that exposure to higher doses is associated with a worse outcome which may be applicable in case of COVID-19. Therefore, health care workers that care for COVID-19 patients are at a particularly high risk as they are more likely to be exposed to a higher number of viral particles, especially in case of a lack of personal protective equipment (PPE).

A correlation between viral load and mortality has been reported. The initial SARS-CoV-2 viral load in nasopharyngeal samples has been decreasing as the pandemic has progressed. The decline in viral load was observed to be associated with a decrease in death rate.

To estimate the viral load, a cycle threshold (Ct) value was provided by the PCR test for each sample—a higher Ct indicated a lower viral load. High, intermediate, and low VL samples respectively were designated to have a Ct value of 25 or under, 26-36, and 37 or over, respectively. A trend in initial VL was observed to coincide with a decrease in the percent of deaths. Almost half of the patients in the high VL group died (45%) compared to 32% and 14% of the intermediate and low VL categories, respectively. Therefore, as the SARS-COV-2 load steadily declined among hospitalized patients there was a corresponding decrease in the percent of deaths over time. Even though confounding variables have not been evaluated, these data suggest an association between initial viral load and mortality.

Exact reasons for a decrease in initial viral load over time have not been determined but the rapid implementation of social distancing measures, lockdown and widespread use of facemasks may have contributed to a decrease in the exposure to the virus.

To further decrease the viral load, a formulation is needed to re-enforce mucosal barrier so that virus that enters the body is prevented from entering the cells and replicating. It is important to note, that people acquire small numbers of viruses from multiple sources (e.g. in a crowd) which may tip the infection over the edge to become symptomatic. Because the infectious dose is probably quite low, it is more likely that individuals will be infected by a single source rather than from multiple sources.

Transmission can take place through small droplets in the air (for example those that are produced after sneezing and which stay in the air for a few seconds). These droplets may be inhaled, or they may land on surfaces. Unfortunately, SARS-COV-2 survives for an extended period of time on most surfaces. It is highly likely, that anyone touching these surfaces and then touching their mouth or nose, are at a risk of being infected. Therefore, hand washing is promoted as a precautionary measure and there is a need to decrease possible contamination of the oral and nasal cavities.

The amount of virus to which an individual is exposed at the start of an infection is referred to as the ‘infectious dose’. For influenza, studies have demonstrated that the higher the initial exposure to a virus or a higher infectious dose, the greater the chances of infection and illness. Studies in mice have also shown that repeated exposure to low doses are as infectious as a single high dose.

As the urgency to develop effective therapeutics against coronaviruses rises, it is important to consider other solutions which decrease the transmission of COVID-19 and provides an individual time to develop an effective immune response against COVID-19. Infectious disease experts have predicted that future outbreaks of novel viruses may continue and hence there is a need to be prepared for a rapid response. Further, there is a need for preventive measures, which decrease transmission and lower viral loads so that an individual has adequate time to mount an effective immune response.

An embodiment the invention described herein relates to compositions and articles used to decrease the viral load SARS-COV-2 on mucosa membranes (for example: oral nasal, and rectal). The objective of this invention is to use mucosal formulations that decease the viral load and create a suboptimal “variolation” effect, by exposing individuals to a smaller number of viral particles and producing a more manageable immune response. Variolation is the level of inoculum below which the immune system is overwhelmed and results in a serious illness. By keeping viral inoculum at a sub-variolation level (which may vary in individuals) the individual successfully fights off the infection, with mild or no clinical illness. This invention is particularly useful in viral diseases in which an individual immune system has a major role to play in the pathogenesis. The formulations described herein decrease the viral inoculum; thereby allowing the immune system of the individuals to clear the viral load.

The invention described herein uses a composition that decreases the viral loads on mucosal surfaces. The invention described herein decreases the viral load to a low (Ct value of 28) or an undetectable amount. Individuals having a low or undetectable viral load do not contribute to the spread SARS-COV-2. The composition interferes with the binding and uptake of beta coronavirus.

The composition contains a significant amount of one or more components which bind to the betacoronavirus and decrease the ability of the virus to bind to host proteins which allow the virus to enter mucosal cells. The composition is effective for interrupting viral binding and uptake. These components are typically less effective if used separately or at low concentrations however the components are highly effective at causing viral membrane leakage.

Compositions and methods are provided for lowering the viral load on mucous membranes. In addition to aqueous formulation, non-solid compositions are applied to mucosal surfaces. In some embodiments, the composition may be non-flowable. In alternative embodiments, the composition may be a liquid which moistens on or around the treatment area. The composition may be in solid state and may be applied to mucous membrane treatment site. The composition may be applied to remain at or near the treatment area. The composition may be removed after being applied temporarily.

A method for bathing and cleansing the mucosal surfaces in the nasal, oral, oropharyngeal cavities decreases the viral inoculum and viral load thereby interfering with the ability of viruses such as betacorona virus (for example COVID-19) to replicate. The formulation is used to decrease the inoculum of other microbials and for routine oral hygienic practices. The method provides use of a liquid mouthwash formulation and/or nasal rinse formulation with polyinterferent properties in an aqueous solution. In some embodiments, the formulation may be in the form of gel, oral strips, chewing gums, or lozenges. The polyinterferent composition damages the membrane of the viral envelop which decreases the binding ability of the virus to the epithelial surface of a host mucosal cell thereby allowing the virus to be washed and ejected from the mucosal surfaces.

Compositions and methods are provided for protecting or preventing or treating a viral attack on the mouth, teeth, gums, lips, oral mucosa. Particularly, but not limited to, biofilm-related conditions in the oral cavity.

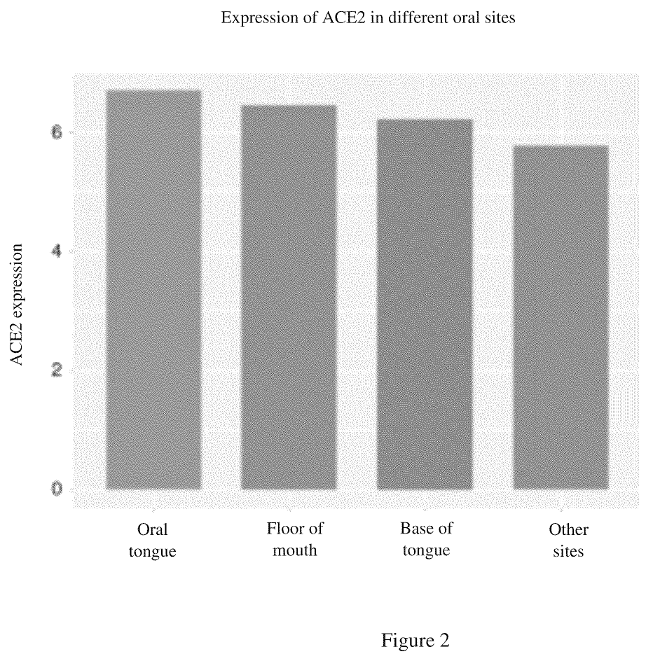

The angiotensin-converting enzyme 2 (ACE2) receptor is a receptor on cell surfaces. SARS-COV-2 virus enters the host cells by binding to the ACE2 receptor. ACE2 receptor is expressed in high concentration in the oral and oropharyngeal cavity, especially in the highly enriched epithelial cells of tongue. See FIG. 2. The polyinterferent composition interferes the binding of SARS-COV-2 viral particles to the ACE2 receptors on the epithelial cells and weakens the viral membrane. Further, the composition interferes with viral fusion and internalization of the viral molecular materials. The polyinterferent composition (PIC) decreases the infectious susceptibility of SARS-COV-2 virus and is as an effective strategy for lowering the inoculum, thereby lowering viral load which allows the subject's natural immune system to combat the virus.

The infectious susceptibility of the oral cavity is high risk for SARS-COV-2 virus. The compositions described herein provide a prevention strategy in dental clinical practice as well as in daily life.

The method for lowering the viral load in a subject using the polyinterferent solution includes the following steps:
1. Rinsing the nasal cavity with polyinterferent solution and/or saline solution and expel mucous, the polyinterferent solution, and/or saline solution for at least about 15 seconds to at least about 60 seconds.
2. Pouring 15-20 ml of unused polyinterferent solution pour into the mouth of the subject and swilling the polyinterferent solution at the posterior pharyngeal wall (back of throat) for at least about 15 seconds to at least about 60 seconds without swallowing the solution.
3. Oscillating the polyinterferent solution over the posterior pharyngeal wall for at least about 15 seconds to at least about 60 seconds.
4. Transferring the polyinterferent solution to the oral cavity and swishing the solution over the surface of the tongue and between cheeks and gums for at least about 15 seconds to at least about 60 seconds.
5. Discarding the polyinterferent from the oral cavity.

In some embodiments, the steps are reiterated at least once. Preferably the nasal cavity is rinsed with the polyinterferent solution prior to rinsing the oral and pharyngeal cavities with the polyinterferent solution. In alternative embodiments, the oral and pharyngeal cavities are rinsed with the polyinterferent solution prior to rinsing the nasal cavity. In some embodiments, the oral, pharyngeal, and nasal cavities are individually rinsed with the polyinterferent solution without a requirement of rinsing all three cavities. The polyinterferent solution is applied to the mucosal membranes of the nasal, oral, and pharyngeal cavities at least once a day and preferably at least twice a day. The pharyngeal cavities are rinsed prior to rinsing the oral cavity thereby avoiding cross contamination.

The invention provides a polyinterferent composition which effectively decreases the load of SARS-COV-2 virus present in the oropharynx.

The polyinterferent composition may be incorporated on tapes or films for direct application or attachment to oropharyngeal, tongue, and buccal surfaces. The polyinterferent composition is adaptive for use in paste, gel or liquids. The toothpaste composition may be a single-phase composition. The toothpaste composition may include conventional additives used in oral care compositions including, but not limited to, fluoride ion sources, anti-calculus agents, anti-tartar agents, buffers, abrasives such as silica, alkali metal bicarbonate salts, thickener materials, humectants, water, surfactants, titanium dioxide, flavoring system, sweetening agents, coloring agents, and mixtures thereof.

The polyinterferent composition is useful for families that are unable to effectively quarantine, especially those that live in multi-generational households.

The invention described herein provides a polyinterferent composition capable of decreasing viral inoculum and thus viral load of SARS-COV-2 virus in an individual. The composition is capable of interfering with the binding, uptake, and/or fusion of the virus to the epithelial membrane. The composition may contain compounds which may include antibacterial agents, such as triclosan, chlorhexidene salts, cetylpyridinium chloride, and domiphen bromide. The composition may also include povidone, carrageenans, griffithsin, thymol, eucalyptol, menthol, methyl salicylate, ethanol, sorbitol, xylitol, zinc chloride, peppermint oil, and/or purified water. The concentration may vary depending on the required strength.

The polyinterferent composition may include chlorhexidine gluconate at a concentration range from about 0.01% to about 0.3%. In alternative embodiments, a separate chlorhexidine gluconate solution is provided in addition to at least one polyinterferent composition, for example a polyinterferent mouthwash and/or a polyinterferent nasal rinse. The chlorhexidine gluconate solution is at a concentration range of about 0.01% to about 0.3%. In some embodiments, chlorhexidine gluconate solution is used as a mouthwash. The solution is gargled at the posterior pharyngeal wall or back of the throat such that the solution contacts the mucosal surfaces of the oropharynx. The chlorhexidine gluconate solution is swished inside the oral cavity thereby contacting the mucosal surfaces of the cheeks, lips, gums, tongue, soft palate, hard palate, and vestibule. The swished and/or gargled solution is then expectorated or expelled out of the oral cavity. In some embodiments, the oropharyngeal cavity and the oral cavity are rinsed with the chlorhexidine gluconate solution prior to rinsing the oropharyngeal cavity and the oral cavity with the polyinterferent composition. In alternative embodiments, oropharyngeal cavity and the oral cavity is rinsed with the polyinterferent composition then the two cavities are rinsed with the chlorhexidine gluconate solution. It is here envisioned that chlorhexidine gluconate solution disrupts the membrane of the viral envelop of the SARS-COV2 virus, which decreases the binding ability of the virus to the epithelial surface of a host mucosal cell thereby allowing the virus to be washed and ejected from the mucosal surfaces.

The polyinterferent composition may include dexamethasone at a concentration range from about 0.05% to about 0.2%. In alternative embodiments, a separate dexamethasone solution may be provided in addition to at least one polyinterferent composition, for example a polyinterferent mouthwash and/or a polyinterferent nasal rinse. The dexamethasone solution is at a concentration range of about 0.05% to about 0.2%. In some embodiments, dexamethasone solution is used as a mouthwash. The solution is gargled at the back of the throat such that the solution contacts the mucosal surfaces of the oropharynx. The solution is then swished inside the oral cavity contacting the mucosal surfaces of the cheeks, lips, gums, tongue, soft palate, hard palate, and vestibule. The swished and/or gargled solution is then expectorated or expelled out of the oral cavity. The solution is gargled at the back of the throat, swished in the oral cavity and then expectorated thereby avoiding cross-contamination of the viral particles with the oral mucosal surfaces. In some embodiments, the dexamethasone solution is used by subjects or patients in the hospital. In some embodiments, the oropharyngeal and the oral cavities are rinsed with the dexamethasone solution prior to rinsing the oropharyngeal cavity and the oral cavity with the polyinterferent composition. In alternative embodiments, oropharyngeal cavity and the oral cavity is rinsed with the polyinterferent composition then the two cavities are rinsed with the dexamthasone solution.

The invention described herein is applied to a mucosal membrane of a subject. The composition is a mouthwash or an oral rinse or a nasal rinse, or a nasal spray, or a chewing gum.

The formulation of the polyinterferent composition in some embodiments of a nasal rinse includes: sodium chloride at a concentration of about 1.5%, iota-carrageenan at a concentration of about 0.06%, xylitol at a concentration of about 5%, benzyl alcohol at a concentration of about 0.2%, potassium sorbate at a concentration of about 0.12%, citric acid at a concentration of about 0.025%, and purified water. The formulation of the polyinterferent composition in some embodiments of a mouthwash includes: iota carrageenan at a concentration about 0.06%, thymol at a concentration of about 0.06%, eucalyptol at a concentration of about 0.0920%, menthol at a concentration of about 0.0420%, methyl salicylate at a concentration of about 0.0600%, ethanol at a concentration of about 26.9%, sorbitol at a concentration of about 14%, xylitol at a concentration of about 5%, peppermint oil at a concentration of about 0.04%, and purified water.

Examples detailing efficacy of the mouthwash formulation for virucidal properties against SARS-COV-2 were performed in a work for hire report conducted under the supervision of Dr. Keith Crawford. The examples conclude that the mouthwash described herein reduced the infectivity of SARS-COV-2 (South African variant) by 2.25 log 10 (99.44%) following a 15 second exposure and by 4.00 log 10 (99.99%) following a 30 second exposure.

The invention now having been fully described, it is further exemplified by the following examples and claims. Those skilled in the art will recognize or be able to ascertain using no more than routine experimentation, numerous equivalents to the specific procedures described herein. Such equivalents are within the scope of the present invention and claims. The contents of all references including issued patents and published patent applications cited in this application are hereby incorporated by reference in their entirety.

### Example 1: Materials and Methods

The host cells used in examples herein were Vero E6 (American Type Culture collection (ATCC) #CRL-1586; green monkey kidney cells, epithelial). A 5% Fetal bovine serum (FBS) was used as an organic soil load (OSL). The growth medium (GM) for the cells included Eagle's Minimum Essential Medium (EMEM), 10% FBS, 1% antibiotics (penicillin, streptomycin, and amphotericin B), and L-glutamine. The maintenance medium (MM) for the cells included EMEM, 2% FBS, 1% antibiotics, and L-glutamine. A Dey-Engley (D/E) Neutralizing broth was used as a product neutralizer. The challenge viral strain used was SARS COV2 isolate hCoV19/South Africa/KRISP-EC-K005321/2020 (Biological and Emerging Infections Resources Program (BEI) Resources #NR-54008). The polyinterferent composition used was a polyinterferent mouthwash solution. The polyinterferent mouthwash is an embodiment of the polyinterferent mouthwash described herein including iota carrageenan at a concentration 0.06%, thymol at a concentration of 0.06%, eucalyptol at a concentration of 0.0920%, menthol at a concentration of 0.0420%, methyl salicylate at a concentration of 0.0600%, ethanol at a concentration of 26.9%, sorbitol at a concentration of 14%, xylitol at a concentration of 5%, peppermint oil at a concentration of 0.04%, and purified water. The polyinterferent composition was used at a final concentration of 90%. The examples were performed at BioScience Laboratories, Inc. (BSLI), Bozeman, Montana, USA. Standard equipment and supplies were used; calibration was in accordance with the standard operating procedure (SOP) of BSLI facility.

### Example 2: Cell Preparation

Vero E6 cells were obtained from American Type Culture collection (ATCC) and were maintained as monolayers in disposable cell culture labware in accordance with BSLI SOP L-2084, “Procedure for subculturing of cells”. The host cell cultures were seeded on 24-well cell culture treated plates. The cells were maintained in a CO₂ incubator having a temperature of about 37° C.±2° C. having CO₂ levels from about 4% to about 6%. Cell monolayers were approximately 80% confluent and less than 48 hours old before inoculation with the virus. The growth medium (GM) was replaced by maintenance medium (MM) prior to inoculation to support virus propagation.

### Example 3: Virus Preparation

The virus was propagated and stored in accordance with BSLI SOP L-2102, “Procedure for production of high-titered virus stock”. The virus was stored at −70° C. On the day of use, aliquots of the stock virus suspension were removed from storage at −70° C. and quickly thawed. FBS was added to the virus suspension to achieve a final concentration of 5%.

### Example 4: Virucidal Suspension Assessment

A 0.5 mL aliquot of virus solution was added to a vial containing 4.5 mL of the polyinterferent composition to achieve a 90% (v/v) final concentration. The virus was exposed to the polyinterferent composition for 15 seconds and 30 seconds using a calibrated minute/second timer. The calibrated minute/second timer was started within +1 second of adding the virus suspension. Immediately after each exposure, the virus/polyinterferent composition suspensions were neutralized in D/E neutralizing broth, mixed thoroughly, and serially diluted in MM. Each dilution was plated in four replicates on cells in multi-well plates. The plates were incubated in a CO₂ incubator from about 7 days to about 14 days at 37° C.±2° C. The cytopathic/cytotoxic effects were monitored using an inverted compound microscope. The assessment was conducted with various positive and negative controls as illustrated in Table 1.

### Example 5: Neutralization Control

A neutralization control sample was established by adding 0.5 mL MM to a vial containing 4.5 mL of the polyinterferent composition. The MM/polyinterferent composition mixture was diluted at a proportion of 1:10 in D/E Neutralizing broth. An aliquot of the virus was added to the neutralized product, thoroughly mixed, and exposed to the neutralized product for at least 30 seconds. The neutralized polyinterferent composition/virus suspensions were diluted 10-fold in MM and each dilution was plated in four replicates on cells in multi-well plates. The plates were incubated in a CO₂ incubator from about 7 days to about 14 days at 37° C.±2° C. The cytopathic/cytotoxic effects were monitored using an inverted compound microscope.

TABLE 1
Virucidal suspension assessment parameters:

| | | Plating |
|---|---|---|
| Parameter | Summary | replicates |
| Virucidal | Virus + Polyinterferent composition → | 4 per group |
| suspension | Exposure → Neutralization → Dilution → | |
| assessment | Plating | |
| Virus control | Virus + Diluent → Exposure → Dilution | 4 per group |
| | → Plating | |
| Cytotoxicity | Diluent + Polyinterferent composition → | 4 per group |
| control | Neutralization → Dilution → Plating | |
| Neutralization | Diluent + Polyinterferent composition → | 4 per group |
| control | Neutralization → Virus inoculation → | |
| | Dilution → Plating | |
| Neutralizer | Virus + Diluent → Neutralization → | 4 per group |
| toxicity control | Dilution → Plating | |
| Cell culture | Maintenance medium | 4 per group |
| control | | |

### Example 6: Neutralizer Toxicity Control

The effect of the neutralizer on virus infectivity was assessed by adding the virus to the D/E neutralizer broth and exposed for at least 30 seconds. The virus/neutralizer broth suspension was diluted 10-fold in MM. Each dilution was plated in four replicates on cells in multi-well plates. The plates were incubated in a CO₂ incubator from about 7 days to about 14 days at 37° C.±2° C. The cytopathic/cytotoxic effects were monitored using an inverted compound microscope.

### Example 7: Cytotoxicity Control

A 0.5 mL aliquot of MM was added to a vial containing 4.5 mL of the polyinterferent composition. The MM/product mixture was neutralized in D/E neutralizing broth, mixed thoroughly, and serially diluted in MM. Each dilution was plated in four replicated on cells in multi-well plates. The plates were incubated in a CO₂ incubator from about 7 days to about 14 days at 37° C.±2° C. The cytopathic/cytotoxic effects were monitored using an inverted compound microscope.

### Example 8: Virus Control

A 0.5 mL aliquot of test virus was added to 4.5 mL of MM and exposed for 30 seconds at ambient temperature. The virus/MM mixture was diluted 10-fold in MM. Each dilution was plated in four replicates on cells in multi-well plates. The plates were incubated in a CO₂ incubator from about 7 days to about 14 days at 37° C.±2° C. The cytopathic/cytotoxic effects were monitored using an inverted compound microscope.

### Example 9: Cell Culture Control

Intact cell culture was used as control for cell culture viability. The GM in the cell control wells was replaced by MM. The plates were incubated in a CO₂ incubator from about 7 days to about 14 days at 37° C.±2° C. The cytopathic/cytotoxic effects were monitored using an inverted compound microscope.

### Example 10: Viral Toxicity Titers

Viral and toxicity titers were expressed as −log₁₀ of the 50% titration end point for infectivity. To calculate the viral titer, a 50% tissue culture infectious dose (TCID₅₀) calculation—the Quantal test (Spearman-Kärber method) was applied.

Log TCID₅₀=−L−d(s−0.5)

In which: L is the −log 10 of the lowest dilution; d is the difference between dilution steps; and s is the sum of proportions of positive wells.

The log₁₀ of infectivity reduction is calculated as follows:

$${Log}_{10} Reduction = ( {log}_{10} {TCID}_{50} of the virus control ) - ( {log}_{10} {TCID}_{50} of the virucidal suspension test )$$

The percent reduction was calculated as follows:

$$\% reduction = [ 1 - ( {TCID}_{50} test / {TCID}_{50} virus control ) ] * 100$$

### Example 11: Test Acceptance Criteria

A valid test required that at least 4 log₁₀ of TCID₅₀ were recovered from the virus control, cells in the cell culture wells were viable and attached to the bottom of the well, the medium was free of contamination in all wells of the plate, for cytotoxicity at least a 3 log₁₀ reduction in titer was demonstrated beyond the cytotoxic level, and the polyinterferent composition was fully neutralized after the timed exposure such that the difference in virus titer for the neutralization control and virus control did not exceed 1.0 log₁₀.

### Example 12: Polyinterferent Mouthwash Reduced Infectivity of SARS-COV-2

Under the conditions of the assessment, the polyinterferent composition, polyinterferent mouthwash reduced the infectivity of SARS-COV-2. The infectivity of SARS CoV2 isolate hCoV19/South Africa/KRISP-EC-K005321/2020 was reduced by 2.25 log₁₀ (99.44%) following a 15 second exposure and by 4.00 log₁₀ (99.99%) following a 30 second exposure. The results of the virucidal assessment of polyinterferent mouthwash are tabulated in Table 2.

TABLE 2
Virucidal assessment of polyinterferent mouthwash results

| | | | | | Neutralizer | | Cell |
|---|---|---|---|---|---|---|---|
| Dilution | Virus | Test | | Neutralization | Toxicity | Cytotoxicity | Control |
| (−Log₁₀) | control | 15 sec | 30 sec | control | Control | control | 0000 |
| −2 | Not tested | ++++ | 0+0+ | Not tested | Not tested | 0000 | N/A |
| −3 | ++++ | ++++ | 0000 | ++++ | Not tested | 0000 | |
| −4 | ++++ | 000+ | 0000 | ++++ | Not tested | 0000 | |
| −5 | ++++ | 0000 | 0000 | ++++ | ++++ | Not tested | |
| −6 | 00++ | 0000 | 0000 | 0+00 | ++00 | Not tested | |
| −7 | 0000 | 0000 | 0000 | 0000 | 0000 | Not tested | |
| TCID₅₀ | 6.00 | 3.75 | 2.00 | 5.75 | 6.00 | ≤1.50 | |
| (Log₁₀) | | | | | | | |
| Log₁₀ | N/A | 2.25 | 4.00 | N/A | | | |
| reduction | | | | | | | |
| Percent | | 99.44% | 99.99% | | | | |
| reduction | | | | | | | |

+: Cytopathic/cytotoxic effect present
0: Cytopathic/cytotoxic effect not detected

1.-32. (canceled)

33. A method for preventing a viral particle of a virus from entering a host cell on a mucosal membrane of a subject by decreasing a viral load of the virus, the method comprising:
administering in a protocol of applying a polyinterferent composition, the composition comprising povidone iodine, iota carrageenan, thymol, eucalyptol, menthol, methyl salicylate, ethanol, and an acceptable buffer, to the mucosal membrane of the subject.

34. The method according to claim 33, the protocol further comprises at least one of the following steps selected from:
irrigating nasal cavities of the subject with saline solution;
rinsing oral and pharyngeal cavities of the subject with the polyinterferent composition;
gargling mouth and throat of the subject with the polyinterferent composition;
administering an antihistamine composition; and
performing respiratory exercises.

35. The method according to claim 34, gargling further comprises a regimen of at least two gargles a day for at least about 10 seconds, about 20 seconds, about 30 seconds, about 40 seconds, about 50 seconds, or about 60 seconds.

36. The method according to claim 34, gargling further comprises contacting deep throat mucosal membranes with the polyinterferent composition.

37. The method according to claim 34, the polyinterferent formulation further comprising: at least one compound selected from: povidone, carrageenan, griffithsin, sorbitol, xylitol, zinc chloride, peppermint oil, and purified water; the formulation further not comprising at least one compound selected from triclosan, chlorhexidene salts, cetylpyridinium chloride, and domiphen bromide; the formulation decreases viral inoculum and viral load of SARS-COV-2 virus in the subject and interferes with the binding, uptake, and/or fusion of the virus to an epithelial membrane of the upper respiratory tract mucosal tissue of the subject.

38. The method according to claim 33, further comprising:
irrigating nasal cavities of the subject with saline solution; rinsing oral and pharyngeal cavities of the subject with the polyinterferent composition; gargling mouth and throat of the subject with the polyinterferent composition; administering an antihistamine composition; and performing respiratory exercises; and optionally reiterating the protocol thereby reducing the amount of inoculum of the virus in the subject.

39.-48. (canceled)

49. The method according to claim 38 wherein reducing the amount of inoculum of the virus further comprises reducing or preventing a viral particle of a SARS-COV-2 virus from entering a host cell on a mucosal membrane of a subject by decreasing viral load.

50. The method according to claim 49, the protocol further comprises
administering an antihistamine composition.

51. The method according to claim 49, the polyinterferent composition is in at least one form selected from: a mouthwash, a nasal spray, and a chewing gum.

52.-55. (canceled)

56. A method for preventing a viral particle of a SARS-COV-2 virus from entering a host cell on a mucosal membrane of a subject by decreasing viral inoculum, the method comprising:
administering a chewing gum comprising an exterior surface encasing a liquid polyinterferent composition, the composition comprising iota carrageenan, thymol, eucalyptol, menthol, methyl salicylate, ethanol, and an acceptable buffer;
breaking the exterior surface of the chewing gum by the subject biting the chewing gum to release the liquid polyinterferent composition into an oral cavity of the subject;
gargling mouth and throat of the subject by contacting deep throat mucosal membranes with the polyinterferent composition; and
expectorating the polyinterferent composition thereby decreasing viral load in the subject.

57. The method according to claim 56, further comprising administering a nasal spray formulation for prevention and amelioration of disease progression caused by the SARS-COV-2 virus, the formulation comprising: iota-carrageenan, sodium chloride, xylitol, benzyl alcohol, potassium sorbate, citric acid, and water.

58. (canceled)

59. The method according to claim 56, further comprising for preventing a viral particle of the SARS-COV-2 virus from entering a host cell on a mucosal membrane of a subject by decreasing viral load, the method comprising:
administering in a protocol of applying a polyinterferent composition, the composition comprising iota-carrageenan, sodium chloride, xylitol, benzyl alcohol, potassium sorbate, citric acid, and an acceptable buffer, to the mucosal membrane of the subject.

60.-64. (canceled)

65. A kit for preventing a viral particle from entering a host cell on a mucosal membrane of a subject by decreasing a viral load, the kit comprising:
a mouthwash comprising a polyinterferent composition comprising iota-carrageenan, sodium chloride, xylitol, benzyl alcohol, potassium sorbate, citric acid, and an acceptable buffer;
a nasal rinse comprising an algal derivative, a salt, and at least one excipient; and
instructions for use.

66. The kit according to claim 65 the instructions further comprise a protocol of applying the mouthwash and the nasal rinse to the mucosal membrane of the subject at least once a day.

67. The kit according to claim 66, the protocol comprises:
irrigating nasal cavities of the subject with the nasal rinse;
rinsing oral and pharyngeal cavities of the subject with the mouthwash; and
gargling mouth and throat of the subject by contacting deep throat mucosal membranes with the mouthwash.

68. The kit according to claim 67, the protocol further comprises at least one of: administering an antihistamine composition, and performing respiratory exercises.

69. The kit according to claim 65, the viral particle is from a SARS-COV-2 virus.

70. The kit according to claim 65 further comprises a test for COVID-19, the test optionally having electronic connectivity to a handheld device.

71. The kit according to claim 65 further comprises at least one of: a chlorhexidine gluconate solution, and a dexamethasone solution.

72. The kit according to claim 65 further comprising a chewing gum.
